(19) United States
(12) Patent Application Publication
(10) Pub. No.: US 2025/0277927 A1
(43) Pub. Date: Sep. 4, 2025

(54) PERIPHERAL LIGHT-EMITTING LINEAR LIGHT GUIDE MEMBER AND METHOD OF MANUFACTURING THE SAME

(71) Applicant: Proterial, Ltd., Tokyo (JP)

(72) Inventors: Oga NORIMASA, Tokyo (JP); Ryuta TAKAHASHI, Tokyo (JP)

(21) Appl. No.: 19/061,170

(22) Filed: Feb. 24, 2025

(30) Foreign Application Priority Data
Feb. 29, 2024 (JP) 2024-030760

(51) Int. Cl. F21V 8/00

(52) U.S. Cl. CPC G02B 6/001

(57) ABSTRACT

A peripheral light-emitting linear light guide member having low manufacturing cost and high uniformity of light intensity is achieved. A peripheral light-emitting optical fiber 3 according to one embodiment includes: an optical fiber 4 including a core 41 exposed from a cladding 42 at one end portion 44; and a light-scattering resin film 5 including a light-transmittable base material 50 and light-scattering particles 51 dispersed in the base material 50, and the light-scattering resin film 5 includes a first region 61, a second region 62, and a third region 63 covering an outer periphery surface of the end portion 44 of the core 41. A minimum film thickness of the second region 62 is equal to or larger than a maximum film thickness of the first region 61, a minimum film thickness of the third region 63 is equal to larger than a maximum film thickness of the second region 62. Each film thickness of the first region 61 and the second region 62 gradually increases in a direction toward the tip end side of the end portion 44, and reaches the maximum film thickness of each region, and a film thickness of the third region 63 gradually increases in the direction toward the tip end side of the end portion 44, and reaches a maximum film thickness of the third region 63, and then, gradually decreases to a predetermined film thickness.

### CROSS-REFERENCE TO RELATED APPLICATION

The application claims foreign priority benefits under 35 U.S.C. § 119 to Japanese Patent Application No. 2024-30760 filed on Feb. 29, 2024, the content of which is hereby incorporated by reference in its entirety.

### TECHNICAL FIELD OF THE INVENTION

The present invention relates to a peripheral light-emitting linear light guide member and a method of manufacturing the same.

### BACKGROUND OF THE INVENTION

A catheter treatment has been known, the catheter treatment being performed by inserting an optical fiber catheter equipped with an optical fiber into a human body's lumen organs (such as esophagus or bowel), blood vessel or heart and treating an affected area by light emitted from a core of the optical fiber. As a peripheral light-emitting linear light guide member for use in such catheter treatment, the Applicant of the present application has suggested a peripheral light-emitting linear light guide member described in Japanese Patent Application Laid-open Publication No. 2022-158714 (Patent Document 1).

The peripheral light-emitting linear light guide member described in the above-described Patent Document includes: an optical fiber with a core exposed by removing a cladding; and a light-scattering member covering an outer periphery surface of the exposed core. The light-scattering member is made of a light-transmittable base material with a refractive index higher than that of the core and light-scattering particles dispersion-mixed with the base material.

In order to enhance uniformity of light intensity in an axial direction, the light-scattering member includes a plurality of layers each with a different ratio of the mixing of the light-scattering particles to the base material, and at least part of each layer overlaps in a radial direction of the core.

In order to form the light-scattering member as described above, solutions of several types each with a different ration of the mixing of the light-scattering particles are prepared, and the solutions are sequentially adhered to the outer circumference of the core, and are cured.

### SUMMARY OF THE INVENTION

The optical fiber catheter for use in catheter treatment is disposable, and its low cost performance is desired. However, in the peripheral light-emitting linear light guide member including the light-scattering member formed as described above, many manhours and long time are required to form the light-scattering member. Therefore, it is not easy to achieve the low cost performance.

A peripheral light-emitting linear light guide member according to one embodiment includes: an optical fiber including a cladding and a core exposed from the cladding at one end portion; and a light-scattering resin film including a light-transmittable base material with a refractive index higher than that of the core and light-scattering particles dispersed in the base material. The light-scattering resin film includes a first region, a second region, and a third region covering an outer periphery surface of the end portion of the core, and the first region, the second region, and the third region are aligned in this order from a base end side to a tip end side of the end portion. A minimum film thickness of the second region is equal to or larger than a maximum film thickness of the first region, and a minimum film thickness of the third region is equal to larger than a maximum film thickness of the second region. Each film thickness of the first region and the second region gradually increases in a direction toward the tip end side of the end portion, and reaches the maximum film thickness of each region. A film thickness of the third region gradually increases in the direction toward the tip end side of the end portion, and reaches a maximum film thickness of the third region, and then, gradually decreases to a predetermined film thickness.

A method of manufacturing a peripheral light-emitting linear light guide member according to one embodiment is a method of manufacturing a peripheral light-emitting linear light guide member including: an optical fiber including a core exposed from a cladding at one end portion; and a light-scattering resin film including a first region, a second region, and a third region covering an outer periphery surface of the end portion of the core. The manufacturing method includes: an optical-fiber processing step of exposing the end portion of the core from the cladding; a film-forming material preparing step of preparing a solution containing light-scattering particles dispersed in a light-transmittable solvent having a refractive index higher than that of the core; an immersing step of immersing the end portion of the core into the solution; and a pull-up step of pulling up the end portion of the core from the solution. The pull-up step includes at least a first step of forming the first region, a second step of forming the second region, and a third step of forming the third region, and the first step, the second step, and the third step are performed in this order. A pull-up start speed in the second step is equal to or higher than a pull-up maximum speed in the first step, and a pull-up start speed in the third step is equal to or higher than a pull-up maximum speed in the second step. A pull-up speed in the first step is gradually increased from a pull-up start speed to the pull-up maximum speed in the first step. The pull-up speed in the second step is gradually increased from the pull-up start speed to the pull-up maximum speed in the second step. The pull-up speed in the third step is gradually increased from the pull-up start speed to a pull-up maximum speed in the third step, and then, is gradually decreased to a predetermined speed.

According to the present invention, a peripheral light-emitting linear light guide member with high uniformity of light intensity can be manufactured at low cost.

### BRIEF DESCRIPTIONS OF THE DRAWINGS

FIG. 1 is a schematic diagram showing a treatment device using a peripheral light-emitting optical fiber as a catheter and a patient as a treatment target.

FIG. 2 is a schematic diagram showing one end portion (tip end portion) of the peripheral light-emitting optical fiber inserted into the body of the patient.

FIG. 3 is a perspective view showing the outer appearance of the tip end portion of the peripheral light-emitting optical fiber.

FIG. 4 is a cross-sectional view showing the sectional structure of the tip end portion of the peripheral light-emitting optical fiber.

FIG. 5 is an explanatory diagram showing a film thickness and a length of each region of a light-scattering resin film.

FIG. 6A is an explanatory diagram showing an optical-fiber processing step.

FIG. 6B is another explanatory diagram showing the optical-fiber processing step.

FIG. 6C is still another explanatory diagram showing the optical-fiber processing step.

FIG. 7 is an explanatory diagram showing a film-forming material preparing step.

FIG. 8 is an explanatory diagram showing an immersing step.

FIG. 9A is an explanatory diagram showing a pull-up step.

FIG. 9B is another explanatory diagram showing the pull-up step.

FIG. 9C is still another explanatory diagram showing the pull-up step.

FIG. 9D is still another explanatory diagram showing a pull-up step.

FIG. 10 is a graph showing a pull-up speed in a first step, a second step, and a third step.

FIG. 11 is a graph showing measurement results of light-emission intensity of the peripheral light-emitting optical fiber.

### DESCRIPTIONS OF THE PREFERRED EMBODIMENTS

Hereinafter, an example of embodiments of the present invention will be described in detail with reference to the accompanying drawings. Note that the same or substantially same configurations and components are denoted by the same reference symbols throughout all the drawings for describing the embodiments. And, the already-described configurations and components will not be repeatedly described in principle.

### <General Description of Treatment Device>

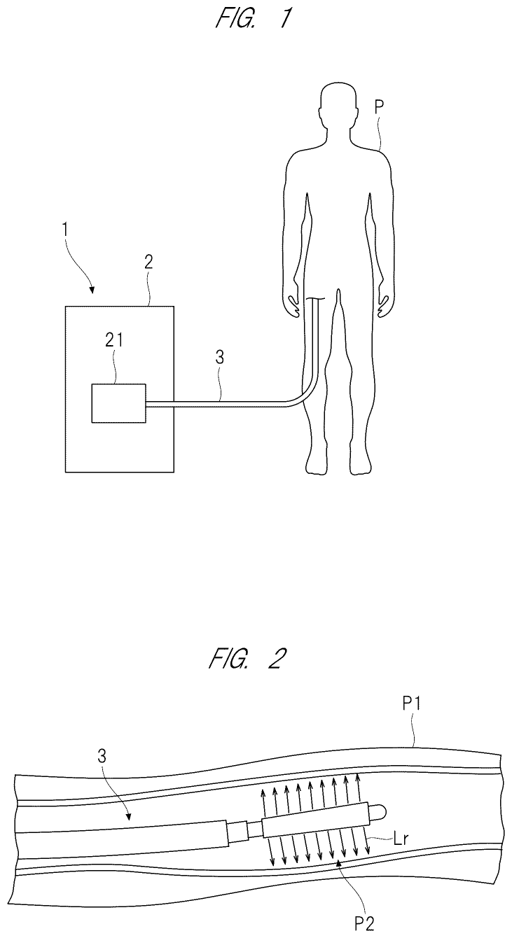

FIG. 1 is a schematic diagram showing a treatment device 1 using a peripheral light-emitting linear light guide member according to the present embodiment as a catheter together with a patient “P” being treated. The treatment device 1 includes a main body 2 and a peripheral light-emitting linear light guide member 3, and a tip end (distal end) of the peripheral light-emitting linear light guide member 3 is inserted into the patient P. The main body 2 includes a light source 21 that emits laser light. The laser light output from the light source 21 included in the main body 2 is incident from a base end (proximal end) of the peripheral light-emitting linear light guide member 3 to the peripheral light-emitting linear light guide member 3. In the following description, the peripheral light-emitting linear light guide member 3 may be referred to as “peripheral light-emitting optical fiber 3”

### <Structure of Peripheral Light-Emitting Linear Light Guide Member>

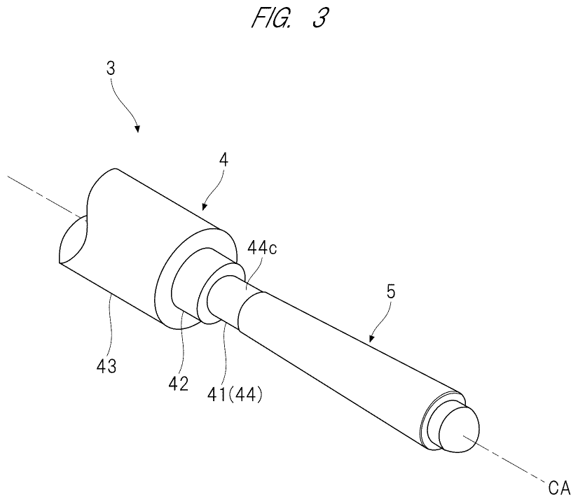

FIG. 2 is a schematic diagram showing one end portion (tip end portion) of the peripheral light-emitting optical fiber 3 being inserted into the body of the patient P. FIG. 2 shows that the blood vessel P1 of the patient P is partially cut out, and that the tip end of the peripheral light-emitting optical fiber 3 is inserted into a blood vessel P1.

The scattered laser light “Lr” emitted from the tip end of the peripheral light-emitting optical fiber 3 irradiates a treated site P2, and activates the drugs previously contained in the treated site P2. This provides intra-vascular laser treatment.

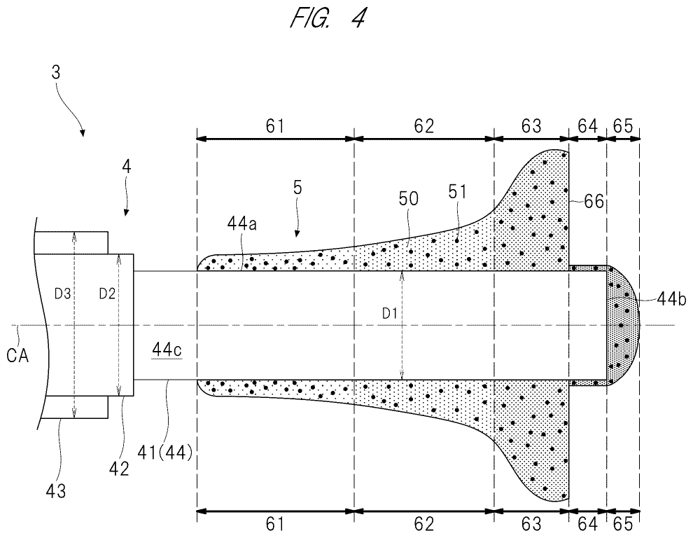

FIG. 3 is a perspective view showing an outer appearance of the tip end of the peripheral light-emitting optical fiber 3. FIG. 4 is a cross-sectional view showing a cross-sectional structure of the tip end of the peripheral light-emitting optical fiber 3. The peripheral light-emitting optical fiber 3 includes: an optical fiber 4, which directs the incident laser light to the treated site P2; and a light-scattering resin film 5 located at one end (tip end) of the optical fiber 4.

Note that FIG. 3 and FIG. 4 exaggeratedly show the film thickness of the light-scattering resin film 5 for convenience of description.

### <<Optical Fiber>>

The optical fiber 4 includes a core 41, a cladding 42, and a sheath 43. At the tip end of the optical fiber 4, the cladding 42 is exposed from the sheath 43, and furthermore, the core 41 is exposed from the cladding 42.

That is, the optical fiber 4 includes the core 41 exposed from the cladding 42 at one end portion. In the following description, the one end portion of the core 41 exposed from the cladding 42 may be referred to as “exposed end portion 44” as distinguished from the other portions of the core 41. Also, the outer periphery surface of the exposed end portion 44 may be referred to as “outer periphery surface 44a”, and the end surface of the exposed end portion 44 may be referred to as “tip end surface 44b”.

The core 41 of the optical fiber 4 is made of quartz glass, and the cladding 42 is made of a polymer. That is, the optical fiber 4 is a quartz-glass optical fiber. Also, the sheath 43 of the optical fiber 4 is made of fluorine-based resin. More specifically, the sheath 43 is made of ETFE (ethylene-tetrafluoroethylene polymer).

The core 41 has a diameter D1 of 200 mm±5 μm, the cladding 42 has a diameter D2 of 225 mm±5 μm, and the sheath 43 has a diameter D3 of 500 mm±30 μm. The refractive index of the core 41 is higher than the refractive index of the cladding 42. Thus, the incident light on the core 41 propagates through the core 41 while being totally reflected at an interface between the core 41 and the cladding 42.

### <<Light-Scattering Resin Film>>

The light-scattering resin film 5 covers a predetermined range of the exposed end portion 44. Note that a root of the exposed end portion 44 has a non-covered portion 44c neither covered with the cladding 42 nor the light-scattering resin film 5.

The light-scattering resin film 5 scatters and emits light emitted from surfaces (the outer periphery surface 44a and the tip end surface 44b) of the exposed end portion 44. The light-scattering resin film 5 includes a light-transmittable base material 50 with a refractive index higher than that of the core 41 and many light-scattering particles 51 that scatter the incident light on the base material 50.

The base material 50 is made of thermosetting resin. In the present embodiment, silicone resin is used. The refractive index of silicone resin is, for example, 1.52. On the other hand, the refractive index of the core 41 is, for example, 1.46.

The light-scattering particles 51 are metal particles that reflect the incident light on the base material 50. In the present embodiment, rutile-type titanium oxide is used. However, the light-scattering particles 51 are not limited to be made of titanium oxide. For example, fine metal powder of aluminum oxide (alumina), silver, copper, iron, or an alloy thereof may be used as the light-scattering particles 51.

The light-scattering particles 51 are dispersion-mixed at a constant ratio over the entire base material 50. The phrase “being dispersion-mixed at a constant ratio” described here means that the light-scattering particles 51 are not unevenly distributed in part of the base material 50 but are mixed so as to be evenly dispersed over the entire base material 50. Note that the light-scattering particles 51 are too small to be recognized by the naked eyes. However, in the attached drawings of the specification, the sizes of the light-scattering particles 51 are exaggeratedly shown.

### <<Each Region of Light-Scattering Resin Film>>

The light-scattering resin film 5 includes a first region 61, a second region 62, a third region 63, a fourth region 64, and a fifth region 65. The first region 61, the second region 62, the third region 63, the fourth region 64, and the fifth region 65 are aligned in this order from the base end side (root side) toward the tip end side of the exposed end portion 44. Also, the first region 61, the second region 62, the third region 63, and the fourth region 64 cover the entire outer circumference of the outer periphery surface 44a of the exposed end portion 44, and the fifth region 65 covers the entire tip end surface 44b of the exposed end portion 44.

The film thicknesses of the light-scattering resin films 5 in the first region 61, the second region 62 and the third region 63 are gradually larger in this order. On the other hand, the film thickness of the light-scattering resin film 5 in the fourth region 64 is substantially constant, and is smaller than a minimum film thicknesses of the same in all of the first region 61, the second region 62 and the third region 63. Also, the fifth region 65 of the light-scattering resin film 5 has a substantially semispherical shape.

Note that the film thickness of the light-scattering resin film 5 is the film thickness of the light-scattering resin film 5 in a direction perpendicular to a center axis line CA of the core 41.

In another viewpoint, each of the first region 61, the second region 62, and the third region 63 of the light-scattering resin film 5 is a “gradually-increasing portion” having a thickness gradually increasing toward the tip end side of the exposed end portion 44. The fourth region 64 of the light-scattering resin film 5 is an “annular small-thickness portion” or “cylindrical small-thickness portion” provided closer to the tip end side than the gradually-increasing portion. The fifth region 65 of the light-scattering resin film 5 is a “tip end-covering portion” provided closer to the tip end side than the “annular small-thickness portion” or “cylindrical small-thickness portion”

### <<Length of Each Region>>

FIG. 5 is an explanatory diagram showing the film thickness and the length of each region of the light-scattering resin film 5. The “length” in the following description means a length along the center axis line CA of the core 41 (length in the axial direction) unless otherwise specified.

A length CL of the exposed end portion 44 of the core 41 is 58.0 mm, and a length L of the light-scattering resin film 5 from a start edge of the first region 61 to an end edge of the third region 63 is 54.0 mm. More specifically, a length L1 of the light-scattering resin film 5 in the first region 61 is 20.0 mm, a length L2 of the same in the second region 62 is 20.0 mm, and a length L3 of the same in the third region 63 is 14.0 mm. Also, a length L4 of the light-scattering resin film 5 in the fourth region 64 is 2.0 mm, and a length L0 of the non-covered portion 44c of the core 41 is 2.0 mm.

As described above, the length L2 of the second region 62 is equal to or larger than the length L1 of the first region 61 (L2≥L1). More specifically, the length L2 of the second region 62 is equal to the length L1 of the first region 61 (L2=L1).

Also, the length L3 of the third region 63 is smaller than the length L1 of the first region 61, and smaller than the length L2 of the second region 62 (L1, L2>L3).

As described above, the light-scattering resin film 5 of the present embodiment satisfies a relation “L1≥L2≥L3”, and also satisfies a relation “L1, L2, L3>L4”.

### <<Film Thickness of First Region>>

The film thickness of the first region 61 gradually increases in a direction toward the tip end side of the exposed end portion 44 to reach a maximum film thickness T1max. As a result, the surface of the first region 61 is tilted with respect to the center axis line CA of the core 41. In other words, the surface of the first region 61 is a tapered surface.

### <<Film Thickness of Second Region>>

The film thickness of the second region 62 gradually increases in the direction toward the tip end side of the exposed end portion 44 to change from a minimum film thickness T2min to a maximum film thickness T2max. As a result, as similar to the surface of the first region 61, the surface of the second region 62 is tilted with respect to the center axis line CA of the core 41. In other words, as similar to the surface of the first region 61, the surface of the second region 62 is a tapered surface.

The minimum film thickness T2min of the second region 62 is equal to or larger than the maximum film thickness T1max of the first region 61 (T2min≥T1max). More specifically, the minimum film thickness T2min of the second region 62 is equal to the maximum film thickness T1max of the first region 61 (T2min=T1max).

Also, the maximum film thickness T2max of the second region 62 is larger than the maximum film thickness T1max of the first region 61 (T2max>T1max).

### <<Film Thickness of Third Region>>

The film thickness of the third region 63 gradually increases in the direction toward the tip end side of the exposed end portion 44 to change from a minimum film thickness T3min to a maximum film thickness T3max. Furthermore, after reaching the maximum film thickness T3max, the film thickness of the third region 63 gradually decreases to a predetermined film thickness T3mid in the direction toward the tip end side of the exposed end portion 44. As a result, the surface of the third region 63 is tilted with respect to the center axis line CA of the core 41. Also, the surface of the third region 63 is a substantially tapered surface as a whole.

The minimum film thickness T3min of the third region 63 is equal to or larger than the maximum film thickness T2max of the second region 62 (T3min≥T2max). More specifically, the minimum film thickness T3min of the third region 63 is equal to the maximum film thickness T2max of the second region 62 (T3min=T2max).

Also, the maximum film thickness T3max of the third region 63 is larger than the maximum film thickness T2max of the second region 62 (T3max>T2max). Furthermore, the maximum film thickness T3max is a maximum value of the film thickness of the light-scattering resin film 5. That is, a maximum film thickness portion of the light-scattering resin film 5 is in the third region 63.

Furthermore, the predetermined film thickness T3mid is smaller than the maximum film thickness T3max of the third region 63, and is larger than the minimum film thickness T3min (T3min<T3mid<T3max).

### <<Film Thickness of Fourth Region>>

The film thickness of the fourth region 64 is smaller than the film thickness of the first region 61 in the entire fourth region 64. Furthermore, as described above, the film thickness of the fourth region 64 is substantially constant in the entire fourth region 64. As a result, the surface of the fourth region 64 is parallel to the center axis line CA of the core 41.

As described above, the light-scattering resin film 5 in the present embodiment satisfies a relation “T1max≤T2min<T2max≤T3min<T3mid<T3max”.

Since the film thickness of the light-scattering resin film 5 changes along the axial direction of the exposed end portion 44 as described above, an annular step surface 66 parallel to the tip end surface 44b of the exposed end portion 44 is provided between the third region 63 and the fourth region 64.

### <Manufacturing Method>

Next, one example of a method of manufacturing the peripheral light-emitting optical fiber 3 will be described. The manufacturing method according to the present embodiment includes at least an optical-fiber processing step, a film-forming material preparing step, an immersing step, and a pull-up step.

Furthermore, the pull-up step includes at least a first step, a second step, a third step, a fourth step, and a fifth step. The first step, the second step, the third step, the fourth step, and the fifth step are performed in this this order.

### <<Optical-Fiber Processing Step>>

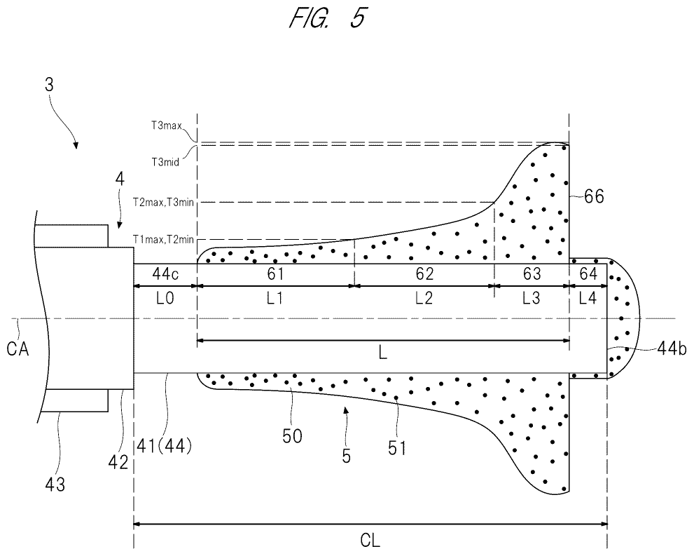

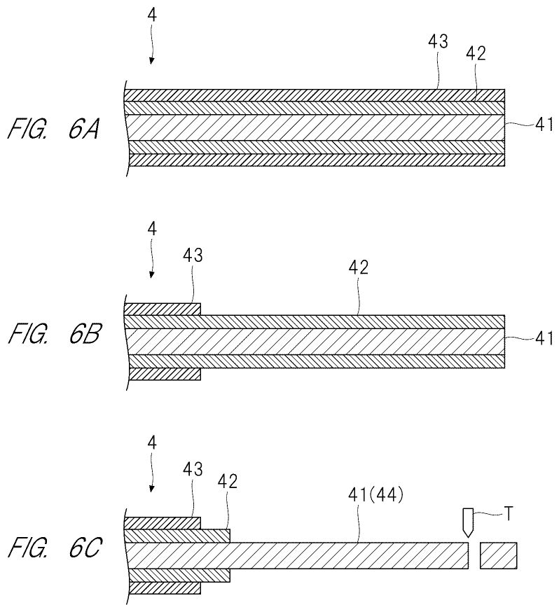

FIGS. 6A, 6B and 6C are explanatory diagrams each showing the optical-fiber processing step. In the optical-fiber processing step, the optical fiber 4 including the core 41, the cladding 42, and the sheath 43 is prepared as shown in FIG. 6A.

Next, as shown in FIG. 6B, the sheath 43 is removed over any length range, and the cladding 42 is exposed. In the present embodiment, the sheath 43 is removed over 75.0 mm.

Then, as shown in FIG. 6C, the cladding 42 is removed to expose the end portion of the core 41 from the cladding 42, and the exposed end portion of the core 41 is cut out so as to have any length.

More specifically, the exposed end portion of the core 41 is cut out so as to have any length longer than an intended length of the light-scattering resin film 5. In the present embodiment, an unnecessary portion (extra length portion) is cut out so that the end portion of the core 41 has a length of 58.0 mm.

By the optical-fiber processing step as described above, the optical fiber 4 having the core 41 including the exposed end portion 44 is obtained.

Note that the cladding 42 can be removed by using, for example, an organic solvent such as acetone. Also, the unnecessary portion of the core 41 can be broken by, for example, making a scratch on the core 41 by using a sharp jig T, and then, applying stress thereon.

### <<Film-Forming Material Preparing Step>>

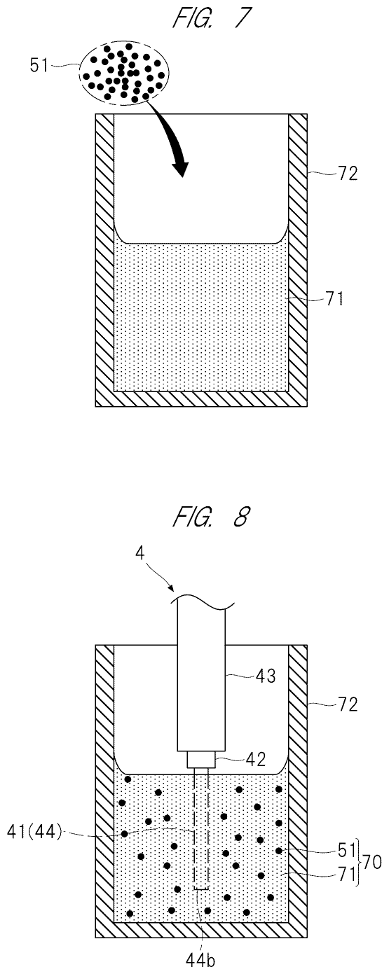

FIG. 7 is an explanatory diagram showing the film-forming material preparing step. In the film-forming material preparing step, the light-scattering particles 51 are dispersion-mixed with a light-transmittable solvent 71 having a refractive index higher than that of the core 41. More specifically, a predetermined amount (for example, 1 mg/mL) of the light-scattering particles 51 is added and dispersed into the liquid solvent 71 contained in a container 72.

As a result, solution 70 (FIG. 8) in which the light-scattering particles 51 of a predetermined ratio are dispersion-mixed with the solvent 71 is obtained. That is, a film-forming material is obtained.

In the present embodiment, note that silicone resin (refractive index is 1.52) is used as the solvent 71. Also, rutile-type titanium oxide is used as the light-scattering particles 51. In the film-forming material preparing step, the viscosity of the solution 70 may be adjusted by adding an organic solvent such as toluene or acetone.

### <<Immersing Step>>

FIG. 8 is an explanatory diagram showing the immersing step. In the immersing step, the exposed end portion 44 of the core 41 is immersed into the solution 70 prepared in the film-forming material preparing step. More specifically, a predetermined length range of the exposed end portion 44 of the core 41 is vertically immersed into the solution 70.

In the present embodiment, the exposed end portion 44 of the core 41 is immersed down to a position of 2.0 mm in the solution 70 from the end surface of the cladding 42. In other words, the exposed end portion 44 of the core 41 is immersed down to a position of 56.0 mm in the solution 70 from the tip end surface 44b.

### <<Pull-Up Step>>

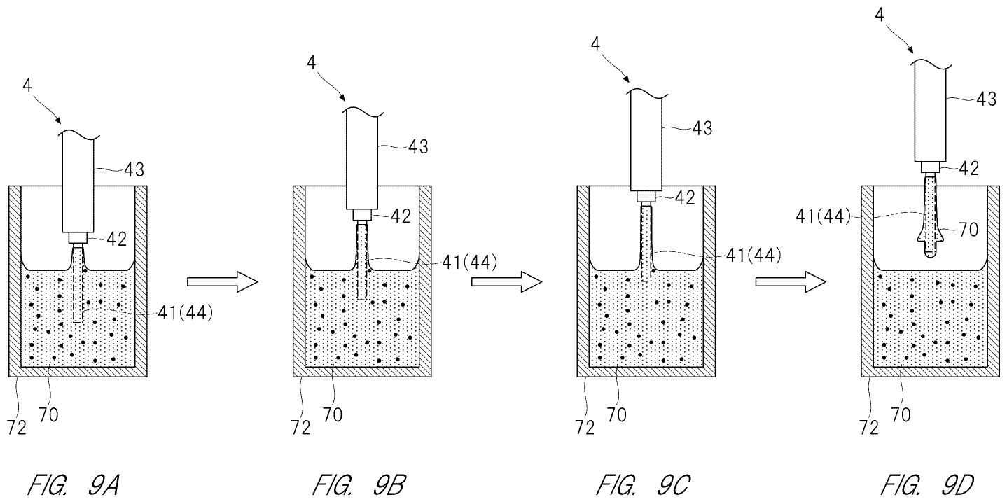

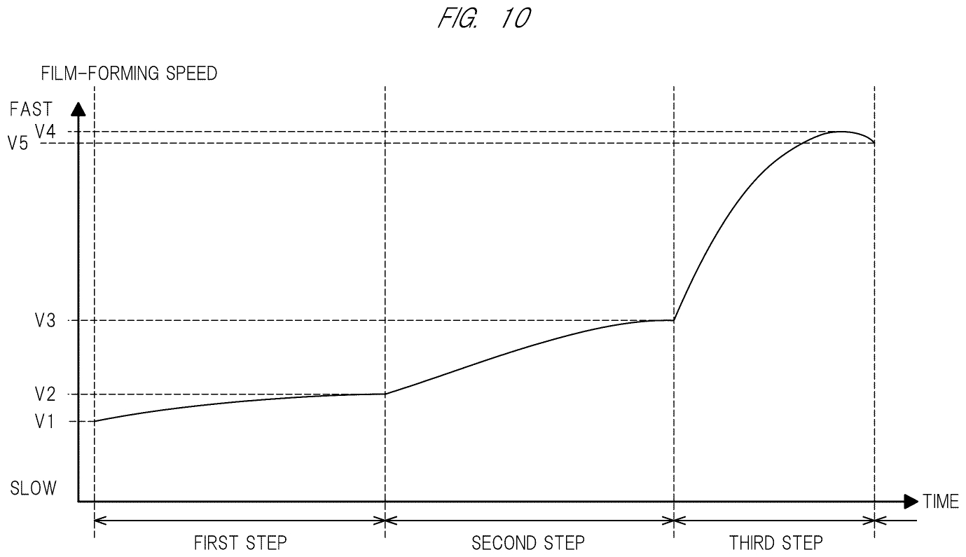

FIGS. 9A, 9B, 9C and 9D are explanatory diagrams each showing the pull-up step. More specifically, FIG. 9A is an explanatory diagram showing a first step of the pull-up step. FIGS. 9B and 9C are explanatory diagrams showing a second step and a third step, respectively. FIG. 9D is an explanatory diagram showing a state of the optical fiber 4 immediately after end of the pull-up step.

In the pull-up step, the exposed end portion 44 of the core 41 is vertically pulled up at a predetermined speed from the solution 70, thereby forming the light-scattering resin film 5. That is, by a dipcoat film-forming method, the light-scattering resin film 5 having a predetermined film thickness and cross-sectional shape is formed. More specifically, the light-scattering resin film 5 including the first region 61 to the fifth region 65 shown in FIGS. 4 and 5 is formed.

In the pull-up step using the dipcoat film-forming method, by controlling the pull-up speed for the exposed end portion 44, a thickness (height) of the solution 70 (film thickness of coating) to be adhered to the surface of the exposed end portion 44 can be controlled.

That is, by controlling the pull-up speed for the exposed end portion 44, the film thickness of each region of the light-scattering resin film 5 can be controlled. In the present step, the thickness of the solution 70 to be adhered to the surface of the exposed end portion 44 can be calculated from the following equation (1).

$$[ Equation 1 ]$$

$$h = \frac{0.94 {( η U )}^{2 / 3}}{{γ}^{1 / 6} \sqrt{ρ g}} \qquad Equation 1$$

Here, terms in Equation 1 represent “h”: the thickness (m) of the solution 70 to be adhered to the surface of the exposed end portion 44, “η”: viscosity (Pa·s) of the solution 70, “U”: pull-up speed (m/s) for the exposed end portion 44, “γ”: surface tension (mN/m) of the solution 70, “p”: density (kg/m³) of the solution 70, “g”: gravitational acceleration (m/s²).

From Equation 1 above, it can be found that the thickness of the solution 70 to be adhered to the surface of the exposed end portion 44 is larger as the pull-up speed for the exposed end portion 44 is higher while the thickness of the solution 70 to be adhered to the surface of the exposed end portion 44 is smaller as the pull-up speed for the exposed end portion 44 is lower.

Therefore, by increasing or decreasing the pull-up speed, the first region 61 to the fourth region 64 each having the above-described film thickness can be formed.

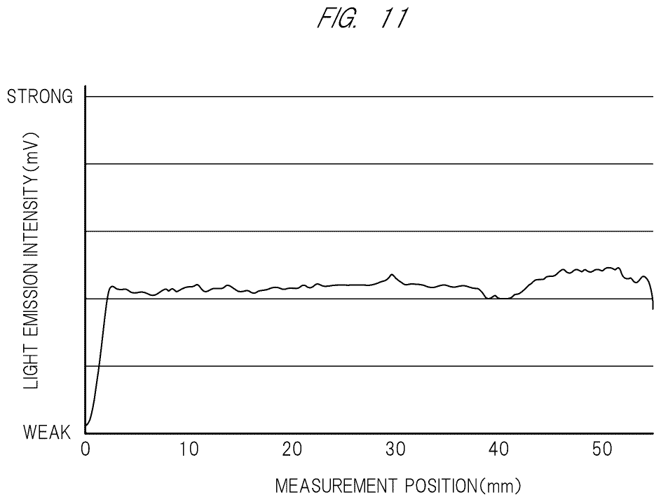

FIG. 10 is a graph showing each pull-up speed in the first step (FIG. 9A), a second step (FIG. 9B), and a third step (FIG. 9C).

### First Step

The first step shown in FIG. 9A is a step of forming the first region 61 shown in FIGS. 4 and 5. As shown in FIG. 5, the film thickness of the first region 61 gradually increases in the direction toward the tip end side of the exposed end portion 44 to reach the maximum film thickness T1max.

Thus, in the first step, the pull-up speed gradually increases from a pull-up start speed V1 in the first step to a pull-up maximum speed V2 in the first step.

More specifically, in the first step, the exposed end portion 44 of the core 41 is vertically pulled up from the solution 70 at a pull-up speed satisfying a cubic equation (“speed=ax³+bx²+cx+d” (terms “a”, “b”, “c”, and “d” are rational numbers)).

### Second Step

The second step of the pull-up step shown in FIG. 9B is a step of forming the second region 62 shown in FIGS. 4 and 5. As shown in FIG. 5, the film thickness of the second region 62 gradually increases in the direction toward the tip end side of the exposed end portion 44 to change from the minimum film thickness T2min to the maximum film thickness T2max.

Also, the minimum film thickness T2min of the second region 62 is equal to the maximum film thickness T1max of the first region 61 (T2min=T1max). Furthermore, the maximum film thickness T2max of the second region 62 is larger than the maximum film thickness T1max of the first region 61 (T2max>T1max).

Thus, in the second step, the pull-up speed that is gradually increased to the pull-up maximum speed V2 in the first step is continuously gradually increased to a pull-up maximum speed V3 in the second step.

More specifically, in the second step, the exposed end portion 44 of the core 41 is vertically pulled up from the solution 70 at a pull-up speed satisfying a cubic equation (speed=ex³+fx²+gx+h (terms “e”, “f”, “g”, and “h” are rational numbers)).

As described above, the pull-up maximum speed V2 in the first step is also a pull-up start speed in the second step. In other words, the pull-up start speed in the second step is equal to or higher than the pull-up maximum speed V2 in the first step.

### Third Step

The third step of the pull-up step shown in FIG. 9C is a step of forming the third region 63 shown in FIGS. 4 and 5. As shown in FIG. 5, the film thickness of the third region 63 gradually increases in the direction toward the tip end side of the exposed end portion 44 to change from the minimum film thickness T3min to the maximum film thickness T3max. Furthermore, the film thickness of the third region 63 reaches the maximum film thickness T3max, and then, gradually decreases in the direction toward the tip end side of the exposed end portion 44 to reach the predetermined film thickness T3mid.

In addition, the minimum film thickness T3min of the third region 63 is equal to the maximum film thickness T2max of the second region 62 (T3min=T2max). Also, the maximum film thickness T3max of the third region 63 is larger than the maximum film thickness T2max of the second region 62 (T3max>T2max). Furthermore, the predetermined film thickness T3mid is smaller than the maximum film thickness T3max and is larger than the minimum film thickness T3min (T3min<T3mid<T3max).

Thus, in the third step, the pull-up speed that is gradually increased to the pull-up maximum speed V3 in the second step is continuously gradually increased to a pull-up maximum speed V4 in the third step. Next, the pull-up speed gradually increased to the pull-up maximum speed V4 is gradually decreased to a predetermined speed V5.

More specifically, in the third step, the exposed end portion 44 of the core 41 is vertically pulled up from the solution 70 at a pull-up speed satisfying a quadratic equation (speed=ix²+jx+k (terms “I”, “j”, and “k” are rational numbers)).

As described above, the pull-up maximum speed V3 in the second step is also the pull-up start speed in the third step. In other words, the pull-up start speed in the third step is equal to or higher than the pull-up maximum speed V3 in the second step.

Also, the predetermined speed V5 is lower than the pull-up maximum speed V4 in the third step and is higher than the pull-up start speed V3 in the third step (pull-up maximum speed in the second step) (V3<V5<V4).

### Fourth Step and Fifth Step

Although not illustrated, the fourth step is a step of forming the fourth region 64 shown in FIG. 4, and the fifth step is a step of forming the fifth region 65 shown in FIG. 4.

As described above, in the entire fourth region 64, the film thickness of the fourth region 64 is smaller than the film thickness of the first region 61, and is constant.

Thus, in the fourth step, the pull-up speed is maintained at a constant speed lower than the pull-up start speed V1 in the first step.

As described above, the fifth region 65 covers the entire tip end surface 44b of the exposed end portion 44, and has a substantially semispherical shape. Thus, in the fifth step, the pull-up speed is adjusted so as to form the fifth region 65 having the shape shown in FIG. 5.

As shown in FIG. 9D, at the time of the end of the pull-up step including the above-described first step to fifth step, the solution 70 having the desired thickness is adhered to the periphery of the exposed end portion 44. Then, when the solution 70 is cured, the light-scattering resin film 5 is formed. Here, the cured solvent 71 becomes the base material 50 of the light-scattering resin film 5.

Note that the viscosity of the silicone resin that is the solvent 71 of the solution 70 greatly changes depending on the temperature. Thus, in the pull-up step, the temperature of the solution 70 may be adjusted by heating means (for example, heater) arranged on the periphery of the container 72 so that the viscosity of the solution 70 becomes a desired viscosity. In the present embodiment, the temperature of the solution 70 is maintained at about 40° C. during the pull-up step.

In the present embodiment, after the pull-up step, heat treatment for cross-linking the solvent 71 (base material 50) is performed.

### <Light-Emission Intensity>

### <<Measuring Method and Measuring Results>>

FIG. 11 is a graph showing measuring results of light-emission intensity of the peripheral light-emitting optical fiber 3 manufactured by the above-described manufacturing method.

In this measurement, laser light output from the light source 21 shown in FIG. 1 was made incident on the core 41 of the optical fiber 4 from the base end side of the peripheral light-emitting optical fiber 3, and the intensity of light emitted in a radial direction from the light-scattering resin film 5 was measured.

For measurement of the light intensity, an optical power meter including a semiconductor sensor that generates a voltage in accordance with the intensity of the received light was used. Also, while the peripheral light-emitting optical fiber 3 is moved in the axial direction by a robot, the intensity of the light emitted from the light-scattering resin film 5 was measured at each 0.4-mm interval.

The vertical axis of the graph shown in FIG. 11 represents the light intensity (the output voltage of the semiconductor sensor included in the optical power meter), and the horizontal axis thereof represents a measuring position of the light intensity (a distance from the start edge of the first region 61 of the light-scattering resin film 5 to the measuring position).

In this measurement, the light intensity was measured to have a substantially same level in a section (hereinafter referred to as “light-emission intensity uniform section”) from a position away by 2.0 mm from the end surface of the cladding 42 to a position away by 52.0 mm therefrom.

More specifically, the light-emission intensity uniformity in the light-emission intensity uniform section was 80%. The light-emission intensity uniformity (%) is an index for indicating the uniformity of the light-emission intensity, and is a value obtained by dividing a minimum value of the light-emission intensity measured in the light-emission intensity uniform section by a maximum value thereof and then multiplying the divided result by 100.

Also, the light-emission efficiency of the peripheral light-emitting optical fiber 3 was 88%. The light-emission efficiency (%) is an index for indicating the light utilization efficiency, and is a value obtained by dividing the intensity of light emitted from the light-scattering resin film 5 by the intensity of light made incident on the core 41 of the optical fiber 4 and then multiplying the divided result by 100.

From this measurement, it has been confirmed that the peripheral light-emitting optical fiber 3 manufactured by the above-described manufacturing method provides the substantially uniform light intensity distribution as a whole.

### Operation and Effect of Present Embodiment

The peripheral light-emitting optical fiber 3 according to the present embodiment provides the substantially uniform light intensity distribution as a whole, because of balance between the decrease in the light intensity in the exposed end portion 44 of the core 41 and the increase in the light-scattering particles 51 contained in the light-scattering resin film 5 covering the exposed end portion 44 of the core 41.

The light intensity in the exposed end portion 44 of the core 41 gradually decreases in a direction from the base end side toward the tip side of the exposed end portion 44. On the other hand, the film thickness of the light-scattering resin film 5 gradually increases in a direction from the base end side to the tip side of the exposed end portion 44.

Here, the solution 70 that is the material of the light-scattering resin film 5 is mixed so that the light-scattering particles 51 are uniformly dispersed. Therefore, when the film thicknesses of the first region 61, the second region 62, and the third region 63 of the light-scattering resin film 5 are equal to one another, the amounts (the number) of the light-scattering particles 51 included in the respective regions are substantially equal to one another.

In this case, by the gradual decrease in the light intensity in the exposed end portion 44, the intensity of light emitted from the light-scattering resin film 5 is also gradually decreased. That is, the uniform light intensity distribution is not obtained.

However, in the peripheral light-emitting optical fiber 3 according to the present embodiment in which the film thickness of the light-scattering resin film 5 gradually increases in the direction from the base end side toward the tip side of the exposed end portion 44, the second region 62 includes more light-scattering particles 51 than that in the first region 61, and the third region 63 includes more light-scattering particles 51 than that in the second region 62.

As a result, the decrease in the light intensity in the exposed end portion 44 is cancelled by the increase in the light diffuse reflection due to the light-scattering particles 51, and the substantially uniform light intensity distribution is obtained as a whole.

Also, in the present embodiment, an annular small-thickness portion is provided closer to the tip end side than the third region 63 having the thickest portion of the light-scattering resin film 5. From another viewpoint, the fourth region 64 positioned closer to the tip end side than the third region 63 has a constant smaller film thickness than that of the first region 61.

The fourth region 64 (annular small-thickness portion) as described above is formed by the fourth step of the pull-up step in which the pull-up speed is maintained at a constant speed lower than the pull-up start speed V1 in the first step.

By the fourth step having the lower pull-up speed than those of the first step to the third step, formation of a large resin ball on the periphery of the tip end surface 44b of the exposed end portion 44 is prevented. If a large resin ball is formed on the periphery of the tip end surface 44b of the exposed end portion 44, the resin ball contains many light-scattering particles 51. Thus, the light intensity in this resin ball is locally strengthened, and there is a risk of decrease in the light intensity distribution uniformity.

That is, the fourth region 64 (annular small-thickness portion) formed by the fourth step has a function of suppressing the decrease in the light intensity distribution uniformity.

Furthermore, in the present embodiment, the film thickness of the third region 63 reaches the maximum film thickness, and then, gradually decreases. From another viewpoint, a gradually-decreasing portion having a gradually-smaller film thickness is formed beyond the thickest portion of the light-scattering resin film 5. As a result, rapid decrease in the light intensity is suppressed, and the light intensity distribution uniformity is further improved. In other words, the gradually-decreasing portion is an extra length portion for suppressing the rapid decrease in the light intensity due to the influence of the annular small-thickness portion.

The present invention is not limited to the above-described embodiments, and various modifications can be made within the scope of the present invention. For example, the film thickness of the light-scattering resin film 5 in each region can be appropriately changed on condition that a predetermined relation is satisfied. Nevertheless, if the peripheral light-emitting linear light guide member 3 is used as the catheter, the tip portion of the peripheral light-emitting linear light guide member 3 to be inserted into the body of the patient P is desirably as thin as possible. From this viewpoint, the total of the maximum film thickness T1max of the first region 61, the maximum film thickness T2max of the second region 62, and the maximum film thickness T3max of the third region 63 is desirably smaller than the diameter D1 of the core 41 (T1max+T2max+T3max<D1).

What is claimed is:

1. A peripheral light-emitting linear light guide member comprising:
an optical fiber including a cladding and a core exposed from the cladding at one end portion; and
a light-scattering resin film including a light-transmittable base material having a refractive index higher than that of the core and light-scattering particles dispersed in the base material,
wherein the light-scattering resin film includes a first region, a second region, and a third region covering an outer periphery surface of the end portion of the core,
the first region, the second region, and the third region are aligned in this order from a base end side to a tip end side of the end portion,
a minimum film thickness of the second region is equal to or larger than a maximum film thickness of the first region,
a minimum film thickness of the third region is equal to larger than a maximum film thickness of the second region,
each film thickness of the first region and the second region gradually increases in a direction toward the tip end side of the end portion, and reaches the maximum film thickness of each region, and
a film thickness of the third region gradually increases in the direction toward the tip end side of the end portion, and reaches a maximum film thickness of the third region, and then, gradually decreases to a predetermined film thickness.

2. The peripheral light-emitting linear light guide member according to claim 1,
wherein the predetermined film thickness is smaller than the maximum film thickness of the third region and larger than the minimum film thickness of the third region.

3. The peripheral light-emitting linear light guide member according to claim 1,
wherein the light-scattering resin film further includes a fourth region positioned closer to the tip end side of the end portion than the third region and covering the outer periphery surface of the end portion and a fifth region positioned closer to the tip end side of the end portion than the fourth region and covering an end surface of the end portion,
the fourth region has a film thickness smaller than the film thickness of the first region over the entire fourth region, and
a step surface parallel to the end surface of the end portion of the core is provided between the third region and the fourth region.

4. The peripheral light-emitting linear light guide member according to claim 1,
wherein a length of the second region is equal to or larger than a length of the first region, and
a length of the third region is smaller than the length of the first region and smaller than the length of the second region.

5. A method of manufacturing a peripheral light-emitting linear light guide member,
the peripheral light-emitting linear light guide member including: an optical fiber including a core exposed from a cladding at one end portion; and a light-scattering resin film including a first region, a second region, and a third region covering an outer periphery surface of the end portion of the core,
the method comprising:
an optical-fiber processing step of exposing the end portion of the core from the cladding;
a film-forming material preparing step of preparing a solution containing light-scattering particles dispersed in a light-transmittable solvent having a refractive index higher than a refractive index of the core;
an immersing step of immersing the end portion of the core into the solution; and
a pull-up step of pulling up the end portion of the core from the solution,
wherein the pull-up step includes at least a first step of forming the first region, a second step of forming the second region, and a third step of forming the third region,
the first step, the second step, and the third step are performed in this order,
a pull-up start speed in the second step is equal to or higher than a pull-up maximum speed in the first step,
a pull-up start speed in the third step is equal to or higher than a pull-up maximum speed in the second step,
a pull-up speed in the first step is gradually increased from a pull-up start speed to the pull-up maximum speed in the first step,
the pull-up speed in the second step is gradually increased from the pull-up start speed to the pull-up maximum speed in the second step, and
the pull-up speed in the third step is gradually increased from the pull-up start speed to a pull-up maximum speed in the third step, and then, is gradually decreased to a predetermined speed.

6. The method of manufacturing the peripheral light-emitting linear light guide member according to claim 5,
wherein the predetermined speed is lower than the pull-up maximum speed in the third step and higher than the pull-up start speed in the third step.

7. The method of manufacturing the peripheral light-emitting linear light guide member according to claim 5,
wherein the light-scattering resin film further includes a fourth region covering the outer periphery surface of the end portion and a fifth region covering an end surface of the end portion,
the pull-up step further includes a fourth step of forming the fourth region to be performed after the third step and a fifth step of forming the fifth region to be performed after the fourth step, and
a pull-up speed in the fourth step is a constant speed lower than the pull-up start speed in the first step.

8. The method of manufacturing the peripheral light-emitting linear light guide member according to claim 5,
wherein the pull-up speed in the first step satisfies a cubic equation (speed=ax³+bx²+cx+d (terms “a”, “b”, “c”, and “d” are rational numbers)),
the pull-up speed in the second step satisfies a cubic equation (speed=ex³+fx²+gx+h (terms “e”, “f”, “g”, and “h” are rational numbers)), and
the pull-up speed in the third step satisfies a quadratic equation (speed=ix²+jx+k (terms “i”, “j”, and “k” are rational numbers)).
